(19) United States
(12) Patent Application Publication
(10) Pub. No.: US 2015/0051461 A1
(43) Pub. Date: Feb. 19, 2015

(54) SYSTEM AND METHOD FOR PERFORMING A REMOTE MEDICAL DIAGNOSIS

(71) Applicant: Xerox Corporation, Norwalk, CT (US)

(72) Inventors: Edul N. DALAL, Webster, NY (US); Wencheng WU, Webster, NY (US); Lalit Keshav MESTHA, Fairport, NY (US)

(73) Assignee: XEROX CORPORATION, Norwalk, CT (US)

(21) Appl. No.: 13/968,794

(22) Filed: Aug. 16, 2013

(51) Int. Cl. A61B 5/024; A61B 5/01; A61B 5/1455; A61B 5/00

(52) U.S. Cl. CPC A61B 5/02433; A61B 5/0064; A61B 5/01; A61B 5/02427; A61B 5/14552
USPC 600/323; 600/473; 600/474; 600/476; 600/479; 600/322

(57) ABSTRACT

What is disclosed is a system and method for performing a medical diagnosis for a subject of interest using a RGB video camera and a spot radiometer in a non-contact, remote sensing environment. In one embodiment, video images are captured using a RGB video camera in real-time of a subject of interest for medical diagnostic purposes. The video images are analyzed to identify a region of exposed skin for which measurements are desired to be obtained. A relative position of a spot radiometer is then adjusted such that the spot radiometer can measure incident radiation at a desired wavelength range from the identified region of exposed skin. The measurements are then used to perform a medical diagnosis for the subject. Various embodiments are disclosed.

### TECHNICAL FIELD

The present invention is directed to systems and methods for performing a medical diagnosis for a patient using a video camera and a spot radiometer in a non-contact, remote sensing environment.

### BACKGROUND

Thermal, multi-band and hyper-spectral cameras have arisen for many different medical diagnostics applications in the healthcare field. They provide more wavelength based information beyond the capabilities of inexpensive RGB or NIR cameras but their usability can be limited by cost, since they are typically more expensive than RGB or NIR video cameras. In addition to being expensive, additional trade-offs between image resolution, number of bands, imaging speed and illumination intensity may be required to lower the cost but may not be possible due to tightly integrated system components. Multi-band cameras with a limited number of wavelength bands typically use filters to provide the band separation. These sacrifice either image resolution by distributing the available pixels between the desired bands or imaging speed by sequentially applying the filters to the entire image. In addition, the filters are fixed at pre-selected bands. Fabry-Perot devices allow the bands to be tunable, but at significantly higher cost. The present invention is directed to performing a medical diagnosis for a subject in a non-contact, remote sensing environment with a lower cost option.

### BRIEF SUMMARY

What is disclosed is a system and method for performing a medical diagnosis for a subject of interest using a video camera and a spot radiometer in a non-contact, remote sensing environment. In one embodiment, video images are captured of a subject of interest in real-time. The images are analyzed to identify an area of interest on a region of exposed skin for which measurements are desired to be obtained for medical diagnostic purposes. The position of a spot radiometer and/or the subject are adjusted such that incident radiation can be measured, at a desired wavelength range, from the identified area of interest. The measurements are then used to perform a medical diagnosis. Features and advantages of the teachings hereof will become readily apparent from the following detailed description and accompanying drawings.

### BRIEF DESCRIPTION OF THE DRAWINGS

The foregoing and other features and advantages of the subject matter disclosed herein will be made apparent from the following detailed description taken in conjunction with the accompanying drawings, in which:

FIG. 1 shows a pair of hands with the left hand having an example area of interest 102 and with the right hand having an example area of interest 103;

FIG. 2 illustrates one embodiment of an example system for performing a remote medical diagnostic in accordance with the teachings hereof;

FIG. 3 illustrates one embodiment of the present method for obtaining a medical diagnosis for a patient utilizing the remote medical diagnostic system of FIG. 2; and

FIG. 4 shows a functional block diagram of one embodiment of an example video processing and control system for performing various aspects of the present system as described with respect to the remote medical diagnostic system of FIG. 2 and the flow diagram of FIG. 3.

### DETAILED DESCRIPTION

What is disclosed is a system and method for performing a medical diagnosis for a subject of interest using a video camera and a spot radiometer in a non-contact, remote sensing environment.

### NON-LIMITING DEFINITIONS

A “video” is a time-varying sequence of images captured of a subject of interest using a video camera capable of acquiring a video signal over at least one data acquisition (imaging) channel. The video may also contain other components such as, audio, time reference signals, and the like.

A “subject of interest” refers to a person for which a medical diagnostic is intended to be performed in accordance with the teachings hereof. Although the term “human”, “person”, or “patient” may be used throughout this text, it should be appreciated that the subject may be something other than a human and should not be viewed as limiting the scope of the appended claims strictly to human beings.

“Skin” protects underlying tissues, internal organs, and other anatomical structures against impact, abrasion, ultraviolet radiation, chemical exposure, to name a few. Skin accounts for approximately 16% of total body weight and comprises three layers, i.e., epidermis, dermis, and hypodermis layers. The epidermis is bloodless and dominated by epithelial cells and relies on diffusion of nutrients and oxygen from capillaries within the dermis layer. Melanocytes in the epidermal layer produce various shades of pigment called melanin which protect underlying tissues from ultraviolet radiation. The dermis layer lies between the epidermis and hypodermis and consists of multiple layers with networks of blood vessels, lymphatic structures, nerves, accessory organs such as hair follicles, sweat glands, etc. The hypodermis layer is dominated by adipose tissue and serves as an insulating boundary for the rest of the body.

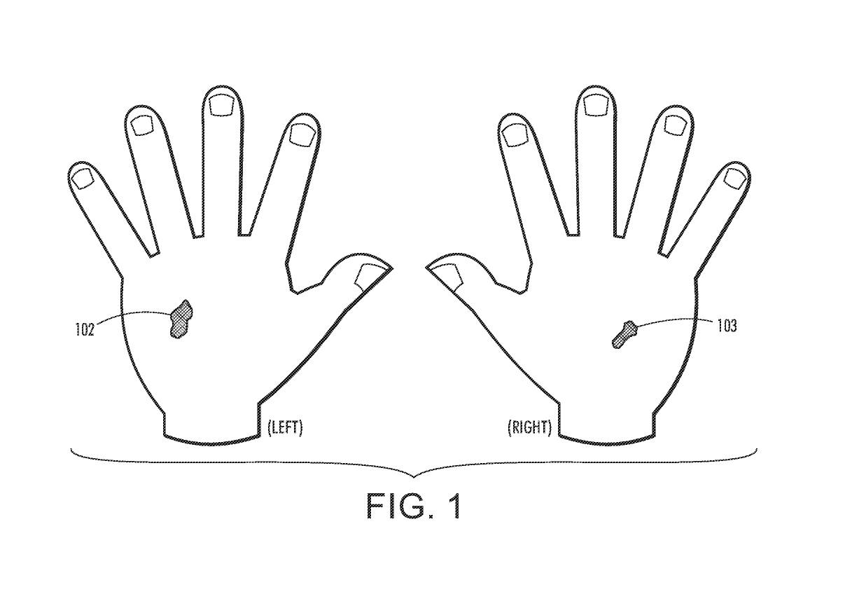

A “region of exposed skin” refers to an unobstructed view of skin wherein an area of interest is identified. FIG. 1 shows a pair of hands with an unobstructed view of a skin surface of a left and right human hand.

An “area of interest” is a surface of the region of exposed skin from which measurements are to be obtained using a spot radiometer. FIG. 1 shows a left hand having a region or an area of interest (lesion 102) and the right hand having an area of interest (lesion 103).

“Identifying an area of interest” means to process the image frames of a video using an image processing technique such that a location of the area of interest can be identified on the region of exposed skin. Image processing techniques include a pixel classification method as disclosed in: “Determining A Total Number Of People In An IR Image Obtained Via An IR Imaging System”, U.S. patent application Ser. No. 12/967,775, by Wang et al., which discloses a ratio method for classifying pixels; “Determining A Number Of Objects In An IR Image”, U.S. patent application Ser. No. 13/086,006, by Wang et al., which discloses a correlation method and a best fitting reflectance method for classifying pixels in an image; and “Determining A Pixel Classification Threshold For Vehicle Occupancy Detection”, U.S. patent application Ser. No. 13/324,308, by Wang et al., which discloses a method for determining a threshold used in pixel classification, all of which are incorporated herein in their entirety by reference. An area of interest on a region of exposed skin that can also be identified using object identification, spatial features, color, shape, pattern recognition, material analysis, texture identification, and facial recognition methods. An area of interest can be manually identified by a technician or medical practitioner using, for example, a mouse or a touchscreen to make a selection thereof from a displayed image. Further, “Automatically Focusing A Spectral Imaging System Onto An Object In A Scene”, U.S. patent application Ser. No. 13/775,665, by Nystrom et al. is incorporated herein in its entirety by reference.

A “video camera” is a device for acquiring a video. In one embodiment, the video camera is a color camera with at least one imaging channel for capturing color values for pixels corresponding generally to the primary visible colors (typically RGB). In another embodiment, the video camera is an infrared video camera with at least one imaging channel for measuring pixel intensity values in the near infrared (NIR) wavelength range. In another embodiment, the video camera comprises a hybrid device capable of capturing both color and infrared video images.

A “spot radiometer” refers to any of a family of devices which measure incident radiation of a desired wavelength range in a single region, i.e., without any imaging. A spot radiometer consists essentially of a single sensor, optionally with a monochromator and optics, to measure incident radiation at a single wavelength band or at several bands using, for example, one or more band-pass filters. The set of spot radiometer devices includes infrared thermometers or pyrometers, ultraviolet radiometers, mid-wave infrared (MWIR) radiometers, long wave infrared (LWIR) radiometers, and spectroradiometers which combine the functions of a spectroscope with the capabilities of a radiometer. In various configurations, spot radiometers may be designed to have a large number of narrow wavelength bands, i.e., spectroradiometers. The spot radiometer is further configured with a means for communicating the obtained measurements to a remote device such as, for example, a server or workstation, over a network using a wired or wireless protocol. Structured and unstructured light sources may be used to illuminate the scene wherein the video camera is actively acquiring video of the subject of interest.

“Adjusting a position” means to manipulate the spot radiometer and/or the subject, relative to one another, so that the spot radiometer can obtain measurements of the area of interest. In one embodiment, the adjustment comprises a movement of the spot radiometer so that a measurement can be obtained while the area of interest is held fixed. A movement of the spot radiometer may be remotely controllable using, for example, a controller which controls a robotic arm capable of moving in any axis of rotation in response to a signal. The spot radiometer may further have a remotely controllable zoom functionality which can zoom-in or zoom-out in response to a signal. In another embodiment, the spot radiometer is held fixed while the subject is instructed, via a visual or audio message, to move the area of interest to a position so that the spot radiometer can obtain the desired measurements. In yet another embodiment, both the spot radiometer and the subject's area of interest are adjusted so that the measurements can be obtained.

“Performing a medical diagnosis” for the subject means using the measurements obtained by the spot radiometer to diagnose a medical condition for the subject relative to the identified area of interest. Medical diagnosis includes analyzing signals to estimate heart rate, respiration rate, oxygen saturation, cardiac arrhythmia, and blood components.

### Example Remote Medical Diagnostic System

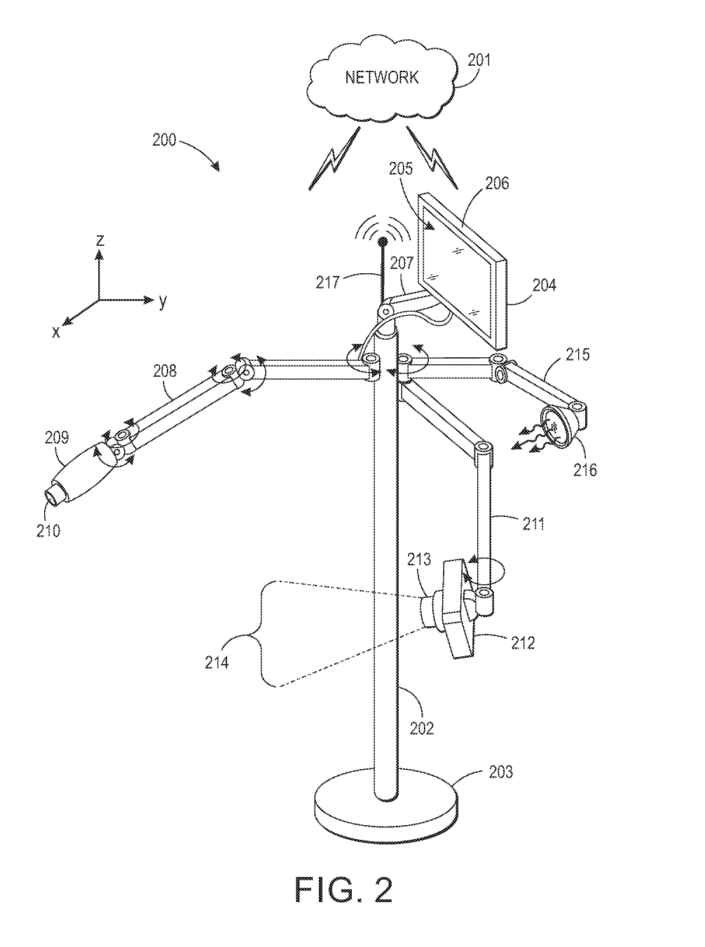

Reference is now being made to FIG. 2 which illustrates one embodiment of an example system for performing a medical diagnostic of an area of interest in accordance with the teachings hereof.

Diagnostic system 200 is shown comprising a support post 202 fixed to support base 203 which sits on a set of wheels (not shown) so that the system can be moved from bed to bed and room to room. In another embodiment, the support post is fixed to a floor, wall, or ceiling of a medical room or healthcare booth. In the embodiment of FIG. 2, support post 202 is shown configured with a plurality of robotic arms. Fixed to a top portion of support post 202 is display device 204 which is shown comprising a display screen 205, such as a LCD or touchscreen device, whereon various messages, images, information, instructions, results, and the like, are displayed for viewing. Display device 204 further is configured with a speaker 206 which, in part, comprises an audio system for playing audio messages and other instructions. Display device 204 is rotatably mounted to robotic arm 207 so that display screen 205 can be turned in any of a plurality of directions for optimum viewing by a user thereof.

Support post 202 further has a robotic support arm 208 whereon a spot radiometer 209 with a telescopic lens 210 is attached. Robotic support arm 208 is remotely controllable so that a position of the spot radiometer affixed to an end thereof is controllably adjustable to move in any of a plurality of directions. In such a manner, a position of the spot radiometer can be adjusted so that a measurement can be obtained of the subject's area of interest. Telescopic lens 210 is also remotely controllable so that the spot radiometer can zoom-in and zoom-out, as needed.

Support post 202 is further configured with another robotic support arm 211 whereon a video camera 212 with a telescopic lens 213 is mounted. The video camera is used herein to capture video images of the subject of interest in the camera's field of view 214. Robotic support arm 211 is also remotely controllable so that a position of the video camera is adjustable to capture video in any of a plurality of directions. Also shown attached to support post 202 is robotic support arm 215 whereon a remotely controllable illuminator 216 is mounted. Illuminator 216, in various embodiments, can project structured or unstructured light, depending on the configuration. Robotic support arms which can rotate in any of a plurality of directions are readily available from different vendors in various streams of commerce. Many such robotic arms are remotely controllable via a wired or wireless communication protocol.

Transmitter 217 effectuates a bi-directional communication with various remote devices over network 201. The transmitter 217 may utilize a wired connection consisting of cables and a hub placed in communication with one or more remote devices over network 201. Any of the devices of the diagnostic system of FIG. 2 may include various elements for communicating with remote devices over network 201. Techniques for placing devices in networked communication are well established. As such, a further discussion as to specific techniques for networking devices has been omitted.

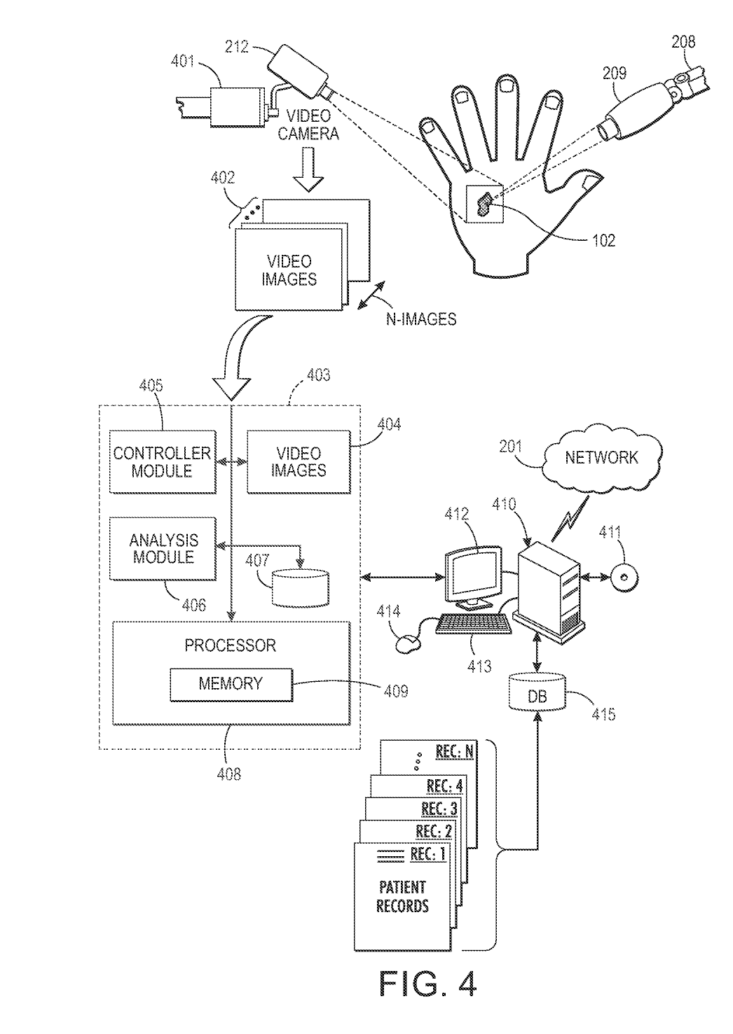

In operation, in one embodiment, a subject of interest seeking a medical diagnosis enters a healthcare booth or room where the system of FIG. 2 is utilized. Instructions displayed on display screen 205 (or by other means such as, for instance, audio) inform the subject where to stand, sit, or lie down. Video images are captured of the subject using video camera 212 and are transmitted over network 201 to a workstation (such as workstation 410 of FIG. 4) which resides in a remote medical facility wherein a technician or medical practitioner views the video and communicates with the subject using one or both of the display 204 and the speaker 206. Medical information is obtained from the subject as the subject answers various questions about their medical issue.

Assume, for discussion purposes, that the subject in the healthcare booth has an unidentified lesion on their left hand (such as that which is shown by way of example at 102 of FIG. 1). The medical practitioner instructs the subject to either stand still or to extend their hand or rest it on a support rest such as an arm of a chair, a surface of a table, or the like, depending on the configuration of the booth. Video of the patient is captured and viewed by the practitioner or processed by automated image processing algorithms to identify and locate the area of interest 102. For example, the image processing algorithm may include a face/hand/body-part detection algorithm followed by a skin color detection algorithm. In the case of face detection, the algorithm may further include a pose detection and/or eye/nose/mouth detection. The additional information about pose and positions of facial features or other body features may be used to guide the adjustment of the relative position between the spot radiometer and the area of interest 102 as discussed below. Upon identifying the area of interest, a position of the spot radiometer is moved, via remotely controllable robotic support arm 208 so that a measurement can be obtained of that area. Alternatively, the subject is instructed to move the area of interest to a particular position so that the spot radiometer can obtain the desired measurements. In another embodiment, both a position of the subject and the spot radiometer are adjusted accordingly so that the desired measurements can be obtained. Measurements obtained by the spot radiometer are communicated, via antenna 217 over network 201, to the medical practitioner where the measurements are analyzed so that a diagnosis can be obtained for the subject with respect to the subject's issues regarding the area of interest. It should be appreciated that the nature and extent of the analysis performed using the measurements will necessarily depend on the medical issues presented by the patient. Therefore, a discussion with respect to a particular analysis has been omitted. It should also be appreciated that the medical diagnostic system of FIG. 2 is one example configuration and that components may be added to the base system which includes a video camera and a spot radiometer, as claimed. For example, the video camera may further include the capability of measuring depth (i.e., distance from the object being imaged to the camera). This capability may be accomplished via use of structured light (e.g. an IR light source) with addition of an appropriate sensor (e.g. an IR sensitive camera) or via an additional video camera with stereo-vision image processing algorithms. An example of a depth enabled video camera is Microsoft Kinect™. If a depth enabled video camera is used, simpler and known image processing methods can be used to identify human features such as face, body, hand, arm, leg etc. Additionally, the depth (distance from the area of interest 102 to the spot radiometer) information can be used to guide the adjustment of the relative position between spot radiometer and the area of interest 102 as discussed above or to correct/calibrate the measurement of the spot radiometer. Furthermore, although the embodiment of FIG. 2 is discussed with respect to obtaining a measurement of a lesion on the subject's left hand, the subject's medical issue may be elsewhere on their body and, as such, the subject may be required to roll up their sleeve or remove their shirt, pants, shoes, etc., so that appropriate measurements can be obtained for the area of interest on the region of exposed skin. For example, for measurement of body temperature, a large area of exposed skin on, e.g., the forehead, may be measured using a spot radiometer operating in the thermal IR band.

### Flow Diagram of One Embodiment

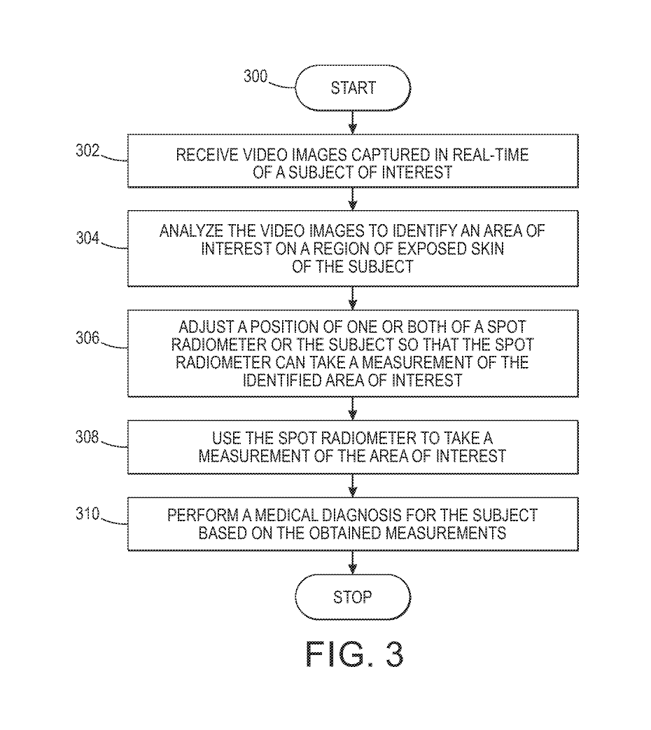

Reference is now being made to the flow diagram of FIG. 3 which illustrates one embodiment of the present method for obtaining a medical diagnosis for a patient in a non-contact, remote sensing environment utilizing the example system of FIG. 2. Flow processing begins at step 300 and immediately proceeds to step 302.

At step 302, receive video images captured in real-time of a subject of interest. Video of the subject of interest can be captured using the video camera 212 of the medical diagnostic system of FIG. 2.

At step 304, analyze the video images to identify an area of interest on a region of exposed skin of the subject. The video can be analyzed utilizing a variety of image processing techniques or by a technician or medical practitioner making a user selection with a mouse or a touchscreen display where the video is displayed.

At step 306, adjust a position of one or both of a spot radiometer and the subject so that the spot radiometer can take a measurement of the area of interest. Adjustment of the spot radiometer 209 can be effectuated using, for instance the remotely controllable robotic arm 208 of FIG. 2. The display device 204 and the audio speaker 206 may be utilized to communicate with the patient to move the area of interest to a desired location so that the measurements can be obtained using the spot radiometer 209.

At step 308, use the spot radiometer to measure incident radiation from a surface of the identified area of interest.

At step 310, perform a medical diagnosis for the subject based upon the obtained measurements. In this embodiment, further processing stops.

The flow diagrams depicted herein are illustrative. One or more of the operations illustrated in the flow diagrams may be performed in a differing order. Other operations may be added, modified, enhanced, or consolidated. Variations thereof are intended to fall within the scope of the appended claims.

### Example Networked System

Reference is now being made to FIG. 4 which shows a functional block diagram of one embodiment of an example video processing and control system 400 for performing various aspects of the present system as described with respect to the medical diagnostic system of FIG. 2 and the flow diagram of FIG. 3.

In FIG. 4, video camera 212 is shown rotatably mounted to a motor comprising, in this embodiment, a step-motor 401 which moves the video camera so that the medical practitioner can focus on the area of interest, i.e., lesion 102, on a region of exposed skin of the subject's left hand. Video images (collectively at 402) are communicated to Control System 403. Video Processor 404 processes the image frames of the video to isolate the region of exposed skin and determine a location of the area of interest in the scene such that a measurement thereof can be obtained by the spot radiometer 209. Video Processor 404 uses any of a variety of image processing methods to facilitate a location and identification of the area of interest in the scene.

Controller Module 405 receives a location of the area of interest in the processed videos and calculates an amount of movement that the spot radiometer 209 needs to be adjusted so that the spot radiometer can take the appropriate measurements. Controller Module 405 may further communicate instructions to the patient to move the region of exposed skin so that a measurement can be obtained by the spot radiometer. In this embodiment, the Video Processor 404 provides the Controller Module with updated information with respect to the subject's movement. The video may be stored to storage device 407. Measurement Analysis Module 406 receives the measurements obtained by the spot radiometer, stores the measurements to storage device 407, and communicates the measurements to the workstation 410 so that a medical diagnosis can be performed for the subject based upon those measurements. Processor 408 retrieves machine readable program instructions from Memory 409 to facilitate the functionality of any of the modules of Control System 403. Processor 408, operating alone or in conjunction with other processors and memory, may also function to process the measurement data depending on the implementation. It should be appreciated that some or all of the functionality of Control System 403 can be incorporated within the video camera 212 or the spot radiometer 209. Control System 403 is shown in communication with a workstation 410.

The computer case of the workstation houses various components such as, for instance, a motherboard with a processor and memory, a communications link such as a network card, a video card, an internal hard drive capable of reading/writing to machine readable media 411 such as a floppy disk, optical disk, CD-ROM, DVD, magnetic tape, and the like, and other software and hardware needed to perform the functionality of a computing system. The workstation further includes a display device 412, such as a CRT, LCD, or touchscreen device, for displaying information, video, measurement data, computational values, patient medical information, results, including distances, locations, and the like. A user can view that information and make a selection from menu options displayed thereon. Keyboard 413 and mouse 414 effectuate a user input or selection. It should be appreciated that the workstation 410 has an operating system and other specialized software configured to display alphanumeric values, menus, scroll bars, dials, slideable bars, pull-down options, selectable buttons, and the like, for entering, selecting, modifying, and accepting information needed for processing video images, and for enabling a medical practitioner to perform a medical diagnosis. Software to configure a user interface or any portion thereof to display/enter/accept data is generally customizable. A user or technician of the workstation may use the graphical user interface to identify regions of interest, set parameters, use a rubber-band box to select image portions and/or regions of images for processing. These selections may be stored and retrieved from storage device 407 and/or computer readable media 411. Default settings and initial parameters can be retrieved from storage device 415, as needed.

In the embodiment of FIG. 4, workstation 410 implements database 415 wherein patient records are stored, manipulated, and retrieved in response to a query. Such records, in various embodiments, take the form of patient medical history stored in association with information identifying the patient and the measurements obtained. Information regarding the region of interest, camera details and settings, wavelengths of the spot radiometers, positioning and locational data, mathematical representations and data values used to perform the medical diagnosis, and the like, may also be stored along with the patient's records. Although the database is shown as an external device, the database may be internal to the workstation mounted, for example, on a hard disk therein. Although shown as a desktop computer, it should be appreciated that the workstation 410 can be a laptop, a mainframe, a client/server, or a special purpose computer such as an ASIC, circuit board, dedicated processor, or the like. The embodiment of the workstation of FIG. 4 is illustrative and may include other functionality known in the arts.

Any of the components of the networked workstation may be placed in communication with Control System 403. Any of the modules and processing units of the Control System 403 can be placed in communication with storage device 415 or computer readable media 411 and may store/retrieve therefrom data, variables, records, parameters, functions, and/or machine readable/executable program instructions, as required to perform their intended functions. Each of the modules of the Control System 403 may be placed in communication with one or more remote devices over network 201. It should be appreciated that some or all of the functionality performed by any of the modules or processing units of system 403 can be performed, in whole or in part, by workstation 410 or by a workstation placed in communication with the Control System 403 over network 201. The embodiment shown is illustrative and should not be viewed as limiting the scope of the appended claims strictly to the configuration shown. In other embodiments, the generated results are provided to a server over network 201 and communicated to multiple user/operators in various diverse locations.

Various modules may designate one or more components which may, in turn, comprise software and/or hardware designed to perform the intended function. A plurality of modules may collectively perform a single function. Each module may have a specialized processor capable of executing machine readable program instructions. A module may comprise a single piece of hardware such as an ASIC, electronic circuit, or special purpose processor. A plurality of modules may be executed by either a single special purpose computer system or a plurality of special purpose computer systems in parallel. Connections between modules include both physical and logical connections. Modules may further include one or more software/hardware modules which may further comprise an operating system, drivers, device controllers, and other apparatuses some or all of which may be connected via a network. It is also contemplated that one or more aspects of the present method may be implemented on a dedicated computer system and may also be practiced in distributed computing environments where tasks are performed by remote devices that are linked through network 201.

The teachings hereof can be implemented in hardware or software using any known or later developed systems, structures, devices, and/or software by those skilled in the applicable art without undue experimentation from the functional description provided herein with a general knowledge of the relevant arts. Moreover, the teachings hereof may be partially or fully implemented in software using object or object-oriented software development environments that provide portable source code that can be used on a variety of computer, workstation, server, network, or other hardware platforms. One or more of the capabilities hereof can be emulated in a virtual environment as provided by an operating system, specialized programs or leverage off-the-shelf computer graphics software such as that in Windows, Java, or from a server or hardware accelerator or other image processing devices.

One or more aspects of the methods described herein are intended to be incorporated in an article of manufacture, including one or more computer program products, having computer usable or machine readable media. The article of manufacture may be included on at least one storage device readable by a machine architecture embodying executable program instructions capable of performing the methodology described herein. The article of manufacture may be shipped, sold, leased, or otherwise provided separately either alone or as part of a product suite or a service.

It will be appreciated that the above-disclosed and other features and functions, or alternatives thereof, may be desirably combined into many other different systems or applications. Various presently unforeseen or unanticipated alternatives, modifications, variations, or improvements therein may become apparent and/or subsequently made by those skilled in the art which are also intended to be encompassed by the following claims. The teachings of any printed publications including patents and patent applications are each separately hereby incorporated by reference in their entirety.

What is claimed is:

1. A method for obtaining a medical diagnosis for a patient in a non-contact, remote sensing environment, the method comprising:
receiving video images captured in real-time of a subject of interest;
analyzing said video images to identify an area of interest on a region of exposed skin of said subject;
adjusting a position of any of: a spot radiometer and said subject, such that a measurement of incident radiation from a surface of said identified area of interest can be obtained using said spot radiometer; and
performing a medical diagnosis for said subject based upon said measurement.

2. The method of claim 1, wherein said spot radiometer comprises any of: an infrared thermometer, a pyrometer, an ultraviolet radiometer, a mid-wave infrared radiometer, a long wave infrared radiometer, and a spectroradiometer.

3. The method of claim 1, wherein said spot radiometer measures incident radiation at any of: a single wavelength band, multiple wavelength bands, and a plurality of narrow wavelength bands.

4. The method of claim 1, wherein analyzing said video images to identify said area of interest comprises any of: pixel classification, spatial features, color, shape, pattern recognition, material analysis, texture identification, and facial recognition methods, and a user selection.

5. The method of claim 1, wherein adjusting a position of said spot radiometer comprises communicating to a controller to direct any of: a zoom level of said spot radiometer onto a determined location, and a movement of said spot radiometer to a determined location.

6. The method of claim 1, wherein adjusting a position of said subject comprises directing said subject to change position by at least one of: playing an audio message over a sound system, displaying a text message on a display device, displaying an image on a display device, and playing a video on a display device.

7. The method of claim 6, wherein directing said subject to change position is based on any of: an estimated pose of said subject, and a difference determined between a desired position and a detected position of said identified area of interest.

8. The method of claim 1, wherein any of: a zoom level, and a movement of said spot radiometer, is remotely controllable.

9. The method of claim 1, wherein any of: a zoom level, and a position of said spot radiometer, is fixed.

10. The method of claim 1, wherein, in advance of said spot radiometer obtaining said measurement, further comprising signaling said spot radiometer to zoom-in on said identified area of interest.

11. The method of claim 1, further comprising communicating with said subject to obtain said subject's medical information.

12. The method of claim 1, further comprising acquiring a depth measurement using a depth sensor.

13. The method of claim 1, wherein said medical diagnosis comprises analyzing signals to estimate any of: heart rate, respiration rate, oxygen saturation, cardiac arrhythmia, and blood components.

14. A system for obtaining a medical diagnosis for a patient in a non-contact, remote sensing environment, the system comprising:
a spot radiometer;
a video camera; and
a processor executing machine readable program instructions for performing:
receiving video images captured in real-time of a subject of interest using said video camera;
analyzing said video images to identify an area of interest on a region of exposed skin of said subject;
adjusting a position of any of: said spot radiometer and said subject, such that a measurement of incident radiation from a surface of said identified area of interest can be obtained using said spot radiometer; and
using said measurement to perform a medical diagnosis for said subject.

15. The system of claim 14, wherein said spot radiometer comprises any of: an infrared thermometer, a pyrometer, an ultraviolet radiometer, a mid-wave infrared radiometer, a long wave infrared radiometer, and a spectroradiometer.

16. The system of claim 14, wherein said spot radiometer measures incident radiation at any of: a single wavelength band, multiple wavelength bands, and a plurality of narrow wavelength bands.

17. The system of claim 14, wherein analyzing said video images to identify said area of interest comprises any of: pixel classification, spatial features, color, shape, pattern recognition, material analysis, texture identification, and facial recognition methods, and a user selection.

18. The system of claim 14, wherein adjusting a position of said spot radiometer comprises communicating to a controller to direct any of: a zoom level of said spot radiometer onto a determined location, and a movement of said spot radiometer to a determined location.

19. The system of claim 14, wherein adjusting a position of said subject comprises directing said subject to change position by at least one of: playing an audio message over a sound system, displaying a text message on a display device, displaying an image on a display device, and playing a video on a display device.

20. The system of claim 19, wherein directing said subject to change position is based on any of: an estimated pose of said subject, and a difference determined between a desired position and a detected position of said identified area of interest.

21. The system of claim 14, wherein any of: a zoom level, and a movement of said spot radiometer, is remotely controllable.

22. The system of claim 14, wherein any of: a zoom level, and a position of said spot radiometer, is fixed.

23. The system of claim 14, wherein, in advance of said spot radiometer obtaining said measurement, further comprising signaling said spot radiometer to zoom-in on said identified area of interest.

24. The system of claim 14, further comprising communicating with said subject to obtain said subject's medical information.

25. The system of claim 14, further comprising acquiring a depth measurement using a depth sensor.

26. The system of claim 14, wherein said medical diagnosis comprises analyzing signals to estimate any of: heart rate, respiration rate, oxygen saturation, cardiac arrhythmia, and blood components.
